(19) United States
(12) Patent Application Publication
(10) Pub. No.: US 2024/0156697 A1
(43) Pub. Date: May 16, 2024

(54) INORGANIC COMPOSITE PIGMENTS ELEVATING UV PROTECTION AND COSMETIC COMPOSITION COMPRISING THE SAME

(71) Applicant: COSMECCA KOREA CO., LTD., Chungcheongbuk-do (KR)

(72) Inventors: Hyun Dae CHO, Cheongju-si (KR); Jong Gun KIM, Icheon-si (KR); Jun Pil HWANG, Seoul (KR); Min Jeung KWON, Seoul (KR); Se Beom CHOI, Namyangju-si (KR); A Young PARK, Incheon (KR); Jae Kyung YU, Yongin-si (KR); Ye Seul LEE, Cheonan-si (KR)

(73) Assignee: COSMECCA KOREA CO., LTD., Chungcheongbuk-do (KR)

(21) Appl. No.: 17/782,182

(22) PCT Filed: Apr. 7, 2022
(86) PCT No.: PCT/KR2022/005063

§ 371 Date: Jun. 3, 2022

(30) Foreign Application Priority Data
May 4, 2021 (KR) 10-2021-0057743

(51) Int. Cl. A61K 8/29; A61K 8/27; A61K 8/34; A61K 8/36; A61K 8/44; A61K 8/58; A61Q 17/04

(52) U.S. Cl. CPC A61K 8/29; A61K 8/27; A61K 8/345; A61K 8/361; A61K 8/44; A61K 8/585; A61Q 17/04; A61K 2800/43; A61K 2800/621; A61K 2800/80

(57) ABSTRACT

An inorganic composite pigment capable of elevating ultraviolet (UV) protection, and a cosmetic composition including the same, in which the inorganic composite pigment includes a core part including a white pigment; and a surface coating material, which is coated on the surface of an external part of the core part, including an inorganic solution that includes the white pigment. Additionally, the white pigment is at least one kind selected from the group consisting of titanium dioxide, zinc oxide, cerium oxide, silica, talc, mica, and borosilicate. The inorganic composite pigment according to the present disclosure capable of elevating a UV protection undergoes surface modification of the pigment through a process of re-coating a white pigment on a white pigment with a high refractive index, thereby elevating the UV protection.

### FIELD OF THE INVENTION

The present disclosure relates to an inorganic composite pigment capable of elevating ultraviolet (UV) protection, and a cosmetic composition containing the same, and more specifically, to an inorganic composite pigment which undergoes surface modification through a process of re-coating a white pigment on a white pigment with a high refractive index thereby elevating its UV protection, and a cosmetic composition containing the same.

### BACKGROUND OF THE INVENTION

UV protection agents used in cosmetics are largely divided into organic UV protection agents and inorganic UV protection agents.

Organic UV protection agents absorb the wavelengths of light, such as UV rays, due to the chemical structure of organic materials with resonance or conjugation structures; however, they can cause skin irritation such as skin trouble, and thus their uses are limited, and as time goes by, they are washed away by sebum, oil fractions, etc. secreted from the skin, affecting their durability. In contrast, inorganic UV protection agents form a protection film that blocks the light itself using an inorganic mineral and scatter UV rays; however, they are stiff when applied due to low spreadability, and unnatural skin appearance occurs due to a white cast phenomenon.

In order to overcome such disadvantages of UV protection agents, along with the recent development of new UV protection agents, agents elevating UV protection that can maximize the effect of a UV protection agent are drawing attention. These materials serve the role of assisting the stabilization of organic UV protection agents, uniform dispersion of inorganic UV protection agents, etc.

However, most synergistic agents elevating UV protection currently distributed in the markets are those having an organic structure, and there are almost no inorganic synergistic agents.

As such, the applicant of the present disclosure has developed an inorganic synergistic agent for blocking UV rays using an inorganic material, which is a composite pigment in which a white pigment (e.g., titanium dioxide) is re-coated on a white pigment with a high refractive index.

### PRIOR ART DOCUMENT

Korean Patent Application Publication No. 10-2007-0110532
Korean Patent No. 10-0602731

### SUMMARY OF THE INVENTION

The present disclosure has been contrived so as to solve the above problems, and a first object thereof is to elevate the effect of UV protection by using a white pigment with a high refractive index (e.g., existing titanium dioxide) as a core and by re-coating the core with a white pigment (e.g., titanium dioxide, etc.), thereby controlling the moving path of light through diffuse reflection on the surface of an inorganic mineral.

Additionally, a second object of the present disclosure is to provide a cosmetic composition containing an inorganic composite pigment, which elevates the UV protection and enables expression of natural skin luminosity and skin tone while correcting the excessive hiding power of a general white pigment and rendering an appropriate hiding power thereto.

Additionally, a third object of the present disclosure is to provide a cosmetic composition containing an inorganic composite pigment, which elevates the UV protection that can provide a long-lasting UV protection effect by absorbing the sebum secreted from the skin due to the synergistic effect of the amount of oil absorption, etc. by re-coating a pigment on the outermost part of the core made of a white pigment.

To achieve the above objects, the inorganic composite pigment according to the present disclosure capable of elevating the UV protection is characterized in that it includes: a core part containing a white pigment; and a surface coating material, which is coated on the surface of an external part of the core part, containing an inorganic solution containing the white pigment.

Additionally, the white pigment is characterized in that it is at least one kind selected from the group consisting of titanium dioxide, zinc oxide, cerium oxide, silica, talc, mica, and borosilicate.

In particular, the white pigment is characterized in that it is contained 20 wt % to 90 wt % relative to the total weight of the inorganic composite pigment; and the inorganic solution is contained 10 wt % to 80 wt % of the total weight of the inorganic composite pigment.

Additionally, the inorganic composite pigment according to the present disclosure capable of elevating the UV protection is characterized in that the surface coating material is coated uniformly or non-uniformly on the core part.

Additionally, the inorganic composite pigment according to the present disclosure capable of elevating the UV protection is characterized in that it has a resonance effect through multiple diffraction and reduction of diffuse reflection on the surface of the surface coating material, and a synergistic effect of UV protection through overlapping and interference of reflected light and transmitted reflected light.

Additionally, the inorganic composite pigment according to the present disclosure capable of elevating the UV protection is characterized in that it has a brightening effect in which the skin tone is naturally realized by controlling light through multiple diffraction, refraction, and reduction of diffuse reflection on the surface of the surface coating material.

Additionally, the inorganic composite pigment according to the present disclosure capable of elevating the UV protection is characterized in that the outside of the surface coating material is: coated with at least one kind of a hydrophobic material selected from the group consisting of silicone, silane, a fluorine-based compound, a metal soap-based compound, a natural surfactant, a polymer, a hydrocarbon-based polymer, higher fatty acid, and higher alcohol; or coated with at least one kind of a hydrophilic material selected from the group consisting of a polyol, polyoxyethylene, and an amino acid-based complex.

In particular, the silicone and silane, which are hydrophobic materials, are at least one kind selected from the group consisting of dimethicone, cyclomethicone, disiloxane, cyclosiloxane, triethoxycaprylylsilane, etc. The fluorine-based compound is at least one kind selected from the group consisting of polyfluoro alkylphosphate ether, perfluoro polymethylisopropyl ether, etc. The metal soap-based compound is at least one kind selected from the group consisting of calcium stearate, sodium stearate, magnesium stearate, etc. The natural surfactant is at least one kind selected from the group consisting of lecithin, hydrogenated lecithin, etc. The polymer is at least one kind selected from the group consisting of collagen, cellulose, xanthan gum, etc. The hydrocarbon-based polymer is at least one kind selected from the group consisting of lauroyl lysine, polyester, polyethylene, polyacrylate, polypropylene, etc. The higher fatty acid is at least one kind selected from the group consisting of stearic acid, myristic acid, palmitic acid, etc. The higher alcohol is at least one kind selected from the group consisting of cetyl alcohol, myristyl alcohol, stearyl alcohol, etc.

Additionally, the polyol, which is a hydrophilic material, is at least one kind selected from the group consisting of glycerin, butylene glycol, dipropylene glycol, propanediol, etc. The polyoxyethylene is at least one kind selected from the group consisting of PEG-10, PEG-40, etc. The amino acid-based complex is at least one kind selected from the group consisting of lysine, arginine, glycine, etc.

Additionally, the inorganic composite pigment according to the present disclosure capable of elevating the UV protection is characterized in that not only a hiding power and a natural skin tone can be realized, but also the make-up can be maintained for a longer period of time due to the increase in the amount of oil absorption through overlapping and interference of reflected light and transmitted reflected light.

Additionally, the cosmetic composition according to the present disclosure contains an inorganic composite pigment capable of elevating the UV protection as an active ingredient.

Additionally, a method for preparing an inorganic composite pigment capable of elevating ultraviolet (UV) protection according to the present disclosure includes:

(a) dispersing titanium dioxide to prepare a white pigment; (b) performing a hydrolysis reaction by adding an aqueous solution of titanium tetrachloride and an aqueous solution of alkali to the dispersed white pigment and then stirring to obtain a titanium hydroxide composite pigment; and (c) cooling and washing the solution in which Step (b) is completed, and then sintering at a temperature between 600° C. and 850° C. to form an oxide.

In particular, Step (a) is characterized in that titanium dioxide is added to distilled water and then stirred at a temperature between 60° C. and 90° C. to prepare a white pigment.

Additionally, Step (b) is characterized in that tetrachloride titanium is dissolved in purified water to prepare an aqueous solution of titanium tetrachloride at a concentration of 10 wt % to 40 wt %; and a hydrolysis reaction is performed by adding an aqueous solution of titanium tetrachloride and an aqueous solution of NaOH at a concentration of 10 wt % to 30 wt % to the dispersed white pigment and then stirring to obtain a titanium hydroxide composite pigment.

Additionally, a method for preparing an inorganic composite pigment capable of elevating ultraviolet (UV) protection according to the present disclosure is characterized in that the method includes:

(a) adding titanium dioxide to distilled water and dispersing titanium dioxide by stirring at a temperature between 60° C. and 90° C. to prepare a white pigment; (b) performing a hydrolysis reaction by adding an aqueous solution of titanium tetrachloride and an aqueous solution of alkali to the dispersed white pigment and then stirring to obtain a titanium hydroxide composite pigment; (c) stirring the solution in which Step (b) is completed for 30 minutes, allowing the solution to stand as-is for 30 minutes, washing the white precipitate generated by standing using distilled water followed by filtration, drying at a temperature between 60° C. and 100° C. to obtain titanium hydroxide composite powder, and sintering the titanium hydroxide composite powder at a temperature between 600° C. and 850° C. to form an oxide of the inorganic pigment on the surface of the white pigment; and (d) in order for the composite pigment to have hydrophobicity, hydrophilicity, or skin affinity, and to enhance the dispersibility of the oxide in the aqueous phase or oil phase, coating the oxide with at least one kind of a hydrophobic material selected from the group consisting of silicone, silane, a fluorine-based compound, a metal soap-based compound, a natural surfactant, a polymer, a hydrocarbon-based polymer, higher fatty acid, and higher alcohol; or coating with at least one kind of a hydrophilic material selected from the group consisting of a polyol, polyoxyethylene, and an amino acid-based complex.

In particular, the silicone and silane, which are hydrophobic materials, are at least one kind selected from the group consisting of dimethicone, cyclomethicone, disiloxane, cyclosiloxane, triethoxycaprylylsilane, etc. The fluorine-based compound is at least one kind selected from the group consisting of polyfluoro alkylphosphate ether, perfluoro polymethylisopropyl ether, etc. The metal soap-based compound is at least one kind selected from the group consisting of calcium stearate, sodium stearate, magnesium stearate, etc. The natural surfactant is at least one kind selected from the group consisting of lecithin, hydrogenated lecithin, etc. The polymer is at least one kind selected from the group consisting of collagen, cellulose, xanthan gum, etc. The hydrocarbon-based polymer is at least one kind selected from the group consisting of lauroyl lysine, polyester, polyethylene, polyacrylate, polypropylene, etc. The higher fatty acid is at least one kind selected from the group consisting of stearic acid, myristic acid, palmitic acid, etc. The higher alcohol is at least one kind selected from the group consisting of cetyl alcohol, myristyl alcohol, stearyl alcohol, etc.

Additionally, the polyol, which is a hydrophilic material, is at least one kind selected from the group consisting of glycerin, butylene glycol, dipropylene glycol, propanediol, etc. The polyoxyethylene is at least one kind selected from the group consisting of PEG-10, PEG-40, etc. The amino acid-based complex is at least one kind selected from the group consisting of lysine, arginine, glycine, etc.

Additionally, the cosmetic composition according to the present disclosure is characterized in that it contains the inorganic composite pigment that elevates the UV protection prepared by the preparation method described above as an active ingredient.

Additionally, the cosmetic composition according to the present disclosure is characterized in that the inorganic composite pigment is contained in an amount of 0.1 wt % to 20 wt % relative to the total weight of the cosmetic composition.

The inorganic composite pigment according to the present disclosure capable of elevating the UV protection uses an inorganic material, in which a white pigment (e.g., titanium dioxide, etc.) is re-coated on a white pigment with a high refractive index; therefore, the inorganic composite pigment has an effect of elevating the UV protection effect by controlling the movement path and reflectivity of light reaching the skin surface through multiple diffraction and a resonance effect on the surface of inorganic minerals.

Additionally, the inorganic composite pigment according to the present disclosure capable of elevating the UV protection has an effect of enabling a natural appearance by exhibiting natural skin luminosity and an effect of skin tone expression through reduction of diffuse reflection.

Additionally, the cosmetic composition containing the inorganic composite pigment according to the present disclosure capable of elevating the UV protection has significantly improved the spreadability and degree of white turbidity (which are the disadvantages of existing inorganic UV protection agents) and has an effect of maintaining a UV protection effect for longer hours by sustaining the cosmetics on the skin for a long period of time because it can absorb the sebum, etc. secreted from the skin with an excellent oil absorption ability compared to general titanium dioxide due to the outermost coating.

### BRIEF DESCRIPTION OF THE DRAWINGS

FIG. 1 is a schematic flow chart illustrating a process for preparing an inorganic composite pigment according to an embodiment of the present disclosure.

FIG. 2 is a schematic diagram illustrating a composite pigment according to an embodiment of the present disclosure, which is in a form consisting of a core part made of a titanium dioxide pigment, and a surface coating material coated on the surface of an external part of the core part; and the moving path of light in the external part.

FIG. 3 is a schematic diagram which illustrates that diffuse reflection is reduced on the surface of an inorganic composite pigment according to an embodiment of the present disclosure due to multiple diffraction effect of light, and UV rays are blocked and skin colors are expressed through the control of light that reaches the skin surface.

FIG. 4 shows scanning electron microscopy (SEM) and transmission electron microscopy (TEM) images of Preparation Example 5 of an inorganic composite pigment according to an embodiment of the present disclosure.

FIG. 5 shows a graph comparing the UV protection index when an inorganic composite pigment according to an embodiment of the present disclosure was applied compared to that of Comparative Example.

### DETAILED DESCRIPTION OF THE INVENTION

Hereinafter, preferred embodiments of the present disclosure will be described in detail with reference to the accompanying drawings. Examples of the present disclosure may be modified in various forms, and the scope of the present disclosure should not be construed as being limited to the Examples described below. These Examples are provided to explain the present disclosure in more detail to those of ordinary skill in the art to which the present disclosure pertains. Accordingly, the shape of each element shown in the drawings may be exaggerated to emphasize a clearer description.

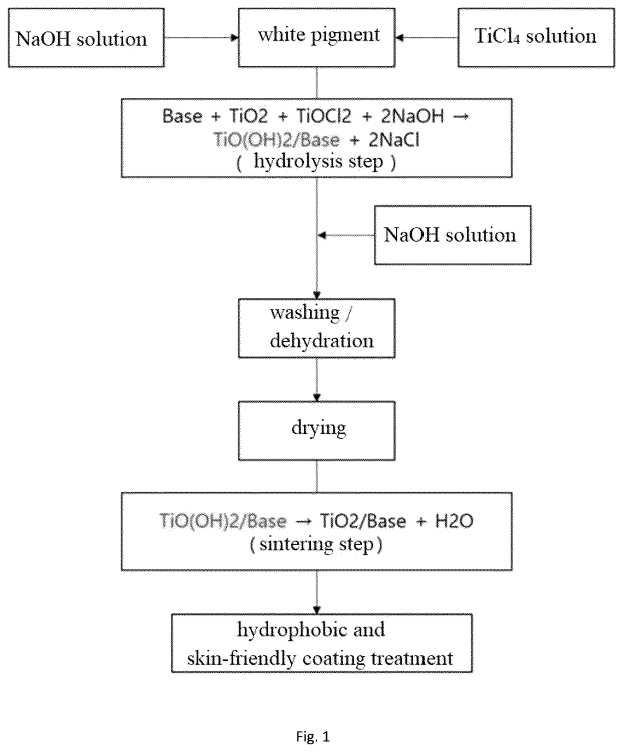

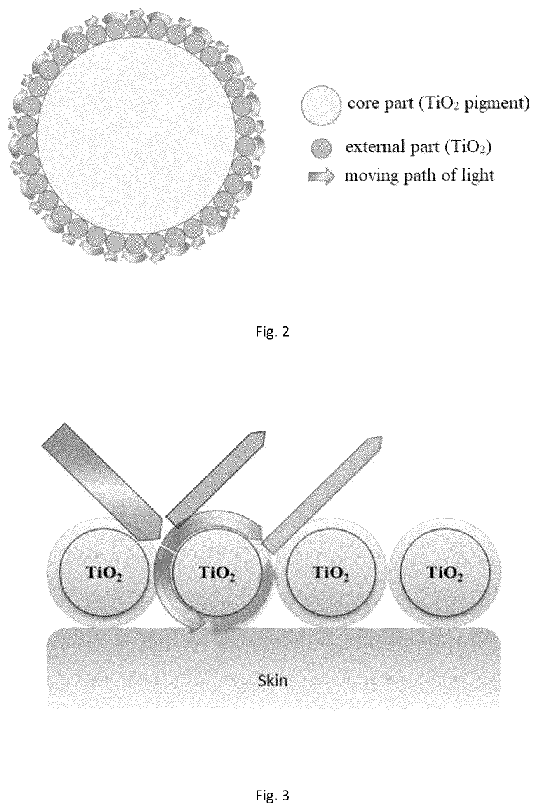

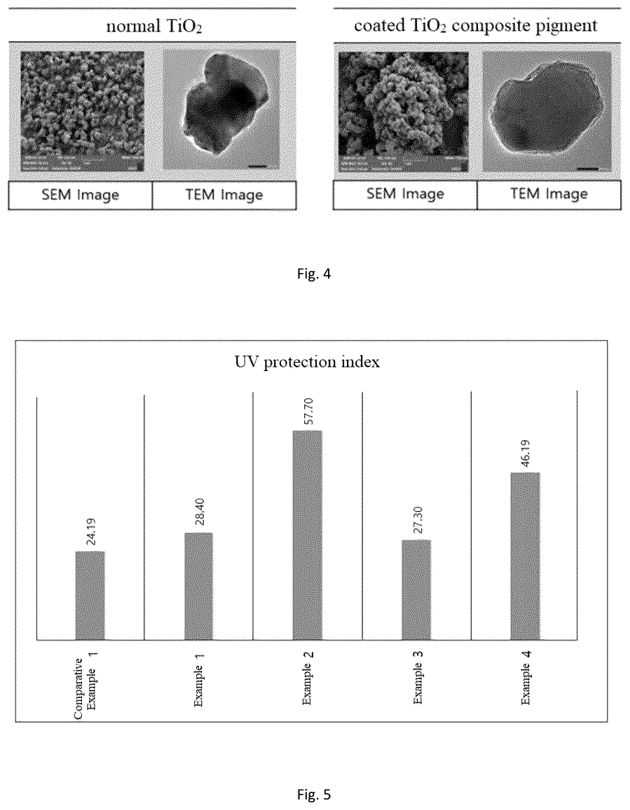

FIG. 1 is a schematic flow chart illustrating a process for preparing an inorganic composite pigment according to an embodiment of the present disclosure; FIG. 2 is a schematic diagram illustrating a composite pigment according to an embodiment of the present disclosure, which is in a form consisting of a core part made of a titanium dioxide pigment, and a surface coating material coated on the surface of an external part of the core part; and the moving path of light in the external part; FIG. 3 is a schematic diagram which illustrates that diffuse reflection is reduced on the surface of an inorganic composite pigment according to an embodiment of the present disclosure due to multiple diffraction effect of light, and UV rays are blocked and skin colors are expressed through the control of light that reaches the skin surface; FIG. 4 shows SEM and TEM images of Preparation Example 5 of an inorganic composite pigment according to an embodiment of the present disclosure; and FIG. 5 shows a graph comparing the UV protection index when an inorganic composite pigment according to an embodiment of the present disclosure was applied compared to that of Comparative Example.

### Preparation Example

Titanium dioxide was added to distilled water and dispersed titanium dioxide by stirring at a temperature of 60° C. to 90° C. to thereby prepare a white pigment, and a titanium hydroxide composite pigment was obtained through hydrolysis using an inorganic solution (e.g., a titanium tetrachloride solution) and an aqueous alkali solution in the dispersed white pigment. The solution completed was stirred for 30 minutes, allowed to stand for 30 minutes, and the white precipitate generated by standing was washed using distilled water followed by filtration, dried at a temperature between 60° C. and 100° C. to thereby obtain titanium hydroxide composite powder. The thus-purified titanium hydroxide composite powder was sintered at a temperature between 600° C. and 850° C. to thereby form an oxide of the inorganic pigment on the surface of the white pigment.

Thereafter, in order for the composite pigment to have hydrophobicity, hydrophilicity, or skin affinity and to enhance the dispersibility of the oxide in the aqueous phase or oil phase, the oxide was coated with at least one kind of a hydrophobic material selected from the group consisting of silicone, silane, a fluorine-based compound, a metal soap-based compound, a natural surfactant, a polymer, a hydrocarbon-based polymer, higher fatty acid, and higher alcohol; or coated with at least one kind of a hydrophilic material selected from the group consisting of a polyol, polyoxyethylene, and an amino acid-based complex.

In particular, the silicone and silane, which are hydrophobic materials, are at least one kind selected from the group consisting of dimethicone, cyclomethicone, disiloxane, cyclosiloxane, triethoxycaprylylsilane, etc. The fluorine-based compound is at least one kind selected from the group consisting of polyfluoro alkylphosphate ether, perfluoro polymethylisopropyl ether, etc. The metal soap-based compound is at least one kind selected from the group consisting of calcium stearate, sodium stearate, magnesium stearate, etc. The natural surfactant is at least one kind selected from the group consisting of lecithin, hydrogenated lecithin, etc. The polymer is at least one kind selected from the group consisting of collagen, cellulose, xanthan gum, etc. The hydrocarbon-based polymer is at least one kind selected from the group consisting of lauroyl lysine, polyester, polyethylene, polyacrylate, polypropylene, etc. The higher fatty acid is at least one kind selected from the group consisting of stearic acid, myristic acid, palmitic acid, etc. The higher alcohol is at least one kind selected from the group consisting of cetyl alcohol, myristyl alcohol, stearyl alcohol, etc.

Additionally, the polyol, which is a hydrophilic material, is at least one kind selected from the group consisting of glycerin, butylene glycol, dipropylene glycol, propanediol, etc. The polyoxyethylene is at least one kind selected from the group consisting of PEG-10, PEG-40, etc. The amino acid-based complex is at least one kind selected from the group consisting of lysine, arginine, glycine, etc.

Preparation Examples of the inorganic composite pigment prepared according to such preparation process are shown in [Table 1] below.

TABLE 1

| Preparation Example | 1 | 2 | 3 | 4 | 5 |
|---|---|---|---|---|---|
| Core White Pigment Content (wt %) | 90 | 80 | 70 | 60 | 50 |
| Inorganic Solution Content in External Part | 10 | 20 | 30 | 40 | 50 |
| (wt %) | | | | | |

With regard to the inorganic composite pigment prepared according to the preparation method above, it was confirmed that a coating surface was formed on the surface of the composite pigment through a scanning electron microscope (SEM) and a transmission electron microscope (TEM). Among them, the results of the coating surface formed on the surface in Preparation Example 5 are shown in FIG. 4.

As shown in FIG. 4, the coated composite pigment formed a wider and non-uniform surface compared to the general titanium dioxide pigment.

As such, the coated composite pigment has a resonance effect through multiple diffraction and reduction of diffuse reflection due to the wide and non-uniform surfaces of the core part and the surface coating material of the coated composite pigment, and an effect of elevating UV protection due to overlapping and interference of reflected light and transmitted reflected light.

Additionally, the coated composite pigment has a brightening effect, in which the skin tone is naturally realized, by controlling light through multiple diffraction and reduction of diffuse reflection on the surfaces of the core part and the surface coating material of the coated composite pigment.

In particular, the white pigment may be contained 20 wt % to 90 wt %, and preferably 30 wt % to 90 wt % relative to the total weight of the inorganic composite pigment. When the white pigment is contained less than 20 wt %, there is a disadvantage in that a concealment force is not formed, whereas when the white pigment is contained more than 90 wt %, there is a disadvantage in that it results in a severe white cast phenomenon.

Additionally, the inorganic solution may be 10 wt % to 80 wt %, and preferably 10 wt % to 70 wt %, relative to the total weight of the inorganic composite pigment. When the inorganic solution is contained less than 10 wt %, there is a disadvantage in that the feeling of use is rough and the transparency is not improved, whereas when the inorganic solution is contained more than 80 wt %, there is a disadvantage in that a concealment force is not formed due to excessive transparency.

### Formulation Example

The results of preparing a UV protection agent containing an inorganic composite pigment according to the present disclosure according to a conventional preparation process are shown in [Table 2] below.

TABLE 2

| | | Content | | | | |
|---|---|---|---|---|---|---|
| | | Comparative | | | | |
| Phase | Comparative | Example 1 | Example 1 | Example 2 | Example 3 | Example 4 |
| Oil Phase | Polyglyceryl-4 Isostearate | 3 | 3 | 3 | 3 | 3 |
| | Hydroxystearate/Sebacate | | | | | |
| | Sorbitan Sesquioleate | 1 | 1 | 1 | 1 | 1 |
| | Dicaprylyl Carbonate | 20 | 20 | 20 | 20 | 20 |
| Pigment | Particulate Titanium | 6 | 6 | 6 | 6 | 6 |
| Part | Dioxide | | | | | |
| | Particulate Zinc Oxide | 12 | 12 | 12 | 12 | 12 |
| | Spherical Pigment | 5 | — | — | — | — |
| | Preparation Example 2 | — | 2.5 | 5 | — | — |
| | Preparation Example 5 | — | — | — | 2.5 | 5 |
| | Disteardimonium | 1 | 1 | 1 | 1 | 1 |
| | Hectorite | | | | | |
| | Dibutyl Adipate | 10 | 10 | 10 | 10 | 10 |
| Aqueous | Magnesium Chloride | 1 | 1 | 1 | 1 | 1 |
| Phase | Butylene Glycol | 7 | 7 | 7 | 7 | 7 |
| | Purified Water | to 100 | to 100 | to 100 | to 100 | to 100 |
| Preservative | Phenoxyethanol | q.s. | q.s. | q.s. | q.s. | q.s. |

A UV protection test was performed to confirm the synergistic effect of UV protection of the composite pigment in a UV protection agent containing the inorganic composite pigment prepared in the formulation example above. In order to confirm UV protection, UV protection 30 minutes after applying 28.6 mg of a UV protection agent to HELIOPLATE HD6 (size: 4.7 cm×4.7 cm, HelioScreen Labs) was confirmed using UV-2000S, a sunscreen analyzer of Labsphere Inc. The test results were confirmed as shown in FIG. 5. The UV protection index was expressed as SPF.

As shown in FIG. 5, it was confirmed that the UV protection index increased when a UV protection agent contained the prepared inorganic composite pigment.

In particular, the inorganic composite pigment may be contained 0.1 wt % to 20 wt %, and preferably 0.5 wt % to 10 wt % relative to the total weight of the UV protection agent. When the inorganic composite pigment is contained less than 0.1 wt %, the UV protection effect is significantly reduced, and additionally, the effect in improving the feeling of use and forming a concealment force, and the synergistic effect of UV protection cannot be expected either. In addition, when the inorganic composite pigment is contained more than 20 wt %, the oily feeling becomes insufficient, making the UV protection agent matte, and it may give a heavy and stiff feeling of use.

Although the present disclosure has been described with reference to the preferred examples mentioned above, other various modifications and variations may be made without departing from the spirit and scope of the present disclosure. Therefore, the disclosed Examples should be considered in an illustrative rather than a restrictive point of view.

1-17. (canceled)

18. An inorganic composite pigment capable of elevating ultraviolet (UV) protection, comprising:
a core part comprising a white pigment; and
a surface coating material, which is coated on the surface of an external part of the core part, comprising an inorganic solution comprising the white pigment.

19. The inorganic composite pigment of claim 18, wherein the white pigment is at least one kind selected from the group consisting of titanium dioxide, zinc oxide, cerium oxide, silica, talc, mica, and borosilicate.

20. The inorganic composite pigment of claim 18, wherein:
the white pigment is contained 20 wt % to 90 wt % relative to the total weight of the inorganic composite pigment; and
the inorganic solution is contained 10 wt % to 80 wt % of the total weight of the inorganic composite pigment.

21. The inorganic composite pigment of claim 18, wherein the surface coating material is coated uniformly or non-uniformly on the core part.

22. The inorganic composite pigment of claim 18, which has a resonance effect through multiple diffraction and reduction of diffuse reflection on the surface of the surface coating material, and a synergistic effect of UV protection through overlapping and interference of reflected light and transmitted reflected light.

23. The inorganic composite pigment of claim 18, which has a brightening effect in which the skin tone is naturally realized by controlling light through multiple diffraction, refraction, and reduction of diffuse reflection on the surface of the surface coating material.

24. The inorganic composite pigment of claim 18, wherein the outside of the surface coating material is:
coated with at least one kind of a hydrophobic material selected from the group consisting of silicone, silane, a fluorine-based compound, a metal soap-based compound, a natural surfactant, a polymer, a hydrocarbon-based polymer, higher fatty acid, and higher alcohol; or
coated with at least one kind of a hydrophilic material selected from the group consisting of a polyol, polyoxyethylene, and an amino acid-based complex.

25. The inorganic composite pigment of claim 24, wherein:
the silicone and the silane are at least one kind selected from the group consisting of dimethicone, cyclomethicone, disiloxane, cyclosiloxane, and triethoxycaprylylsilane;
the fluorine-based compound is at least one kind selected from the group consisting of polyfluoro alkylphosphate ether and perfluoro polymethylisopropyl ether;
the metal soap-based compound is at least one kind selected from the group consisting of calcium stearate, sodium stearate, and magnesium stearate;
the natural surfactant is at least one kind selected from the group consisting of lecithin and hydrogenated lecithin;
the polymer is at least one kind selected from the group consisting of collagen, cellulose, and xanthan gum;
the hydrocarbon-based polymer is at least one kind selected from the group consisting of lauroyl lysine, polyester, polyethylene, polyacrylate, and polypropylene;
the higher fatty acid is at least one kind selected from the group consisting of stearic acid, myristic acid, and palmitic acid; and
the higher alcohol is at least one kind selected from the group consisting of cetyl alcohol, myristyl alcohol, and stearyl alcohol.

26. The inorganic composite pigment of claim 24, wherein:
the polyol is at least one kind selected from the group consisting of glycerin, butylene glycol, dipropylene glycol, and propanediol;
the polyoxyethylene is at least one kind selected from the group consisting of PEG-10 and PEG-40; and
the amino acid-based complex is at least one kind selected from the group consisting of lysine, arginine, and glycine.

27. A cosmetic composition comprising the inorganic composite pigment capable of elevating ultraviolet (UV) protection of claim 18 as an active ingredient.

28. The cosmetic composition of claim 27, wherein the inorganic composite pigment is contained 0.1 wt % to 20 wt % relative to the total weight of the cosmetic composition.

29. A method for preparing an inorganic composite pigment capable of elevating ultraviolet (UV) protection, comprising the following steps:
(a) dispersing titanium dioxide to prepare a white pigment;
(b) performing a hydrolysis reaction by adding an aqueous solution of titanium tetrachloride and an aqueous solution of alkali to the dispersed white pigment and then stirring to obtain a titanium hydroxide composite pigment; and
(c) cooling and washing the solution in which Step (b) is completed, and then sintering to form an oxide.

30. The method of claim 29, wherein in Step (a), titanium dioxide is added to distilled water and then stirred at a temperature between 60° C. and 90° C. to prepare a white pigment.

31. The method of claim 29, wherein in Step (b), tetrachloride titanium is dissolved in purified water to prepare an aqueous solution of titanium tetrachloride at a concentration of 10 wt % to 40 wt %; and a hydrolysis reaction is performed by adding an aqueous solution of titanium tetrachloride and an aqueous solution of NaOH at a concentration of 10 wt % to 30 wt % to the dispersed white pigment and then stirring to obtain a titanium hydroxide composite pigment.

32. A method for preparing an inorganic composite pigment capable of elevating ultraviolet (UV) protection, comprising the following steps:
(a) adding titanium dioxide to distilled water and dispersing titanium dioxide by stirring at a temperature between 60° C. and 90° C. to prepare a white pigment;
(b) performing a hydrolysis reaction by adding an aqueous solution of titanium tetrachloride and an aqueous solution of alkali to the dispersed white pigment and then stirring to obtain a titanium hydroxide composite pigment;
(c) stirring the solution in which Step (b) is completed for 30 minutes, allowing the solution to stand as-is for 30 minutes, washing the white precipitate generated by standing using distilled water followed by filtration, drying at a temperature between 60° C. and 100° C. to obtain titanium hydroxide composite powder, and sintering the titanium hydroxide composite powder at a temperature between 600° C. and 850° C. to form an oxide of the inorganic pigment on the surface of the white pigment; and
(d) coating the oxide with at least one kind of a hydrophobic material selected from the group consisting of silicone, silane, a fluorine-based compound, a metal soap-based compound, a natural surfactant, a polymer, a hydrocarbon-based polymer, higher fatty acid, and higher alcohol; or coating with at least one kind of a hydrophilic material selected from the group consisting of a polyol, polyoxyethylene, and an amino acid-based complex.

33. A cosmetic composition comprising the inorganic composite pigment capable of elevating ultraviolet (UV) protection prepared by the method of claim 29 as an active ingredient.

34. The cosmetic composition of claim 33, wherein the inorganic composite pigment is contained in an amount of 0.1 wt % to 20 wt % relative to the total weight of the cosmetic composition.

35. A cosmetic composition comprising the inorganic composite pigment capable of elevating ultraviolet (UV) protection prepared by the method of claim 32 as an active ingredient.

36. The cosmetic composition of claim 35, wherein the inorganic composite pigment is contained in an amount of 0.1 wt % to 20 wt % relative to the total weight of the cosmetic composition.
